(19) United States
(12) Patent Application Publication
(10) Pub. No.: US 2024/0341594 A1
(43) Pub. Date: Oct. 17, 2024

(54) IDENTIFICATION OF VISION DEGRADING SYMPTOMATIC FLOATERS

(71) Applicant: Alcon Inc., Fribourg (CH)

(72) Inventor: Zsolt Bor, San Clemente, CA (US)

(21) Appl. No.: 18/633,987

(22) Filed: Apr. 12, 2024

Related U.S. Application Data
(60) Provisional application No. 63/495,970, filed on Apr. 13, 2023.

(51) Int. Cl. A61B 3/12; A61B 3/00; A61B 3/10; A61B 3/14

(52) U.S. Cl. CPC A61B 3/12; A61B 3/0058; A61B 3/0091; A61B 3/1025; A61B 3/145

(57) ABSTRACT

Particular embodiments disclosed herein include projecting an orientation grid into an eye of a patient. A computer system captures one or more images of a retina of an eye of the patient, such as using an SLO, while projecting the orientation grid into the eye of the patient. A fixation grid may also be displayed with the patient being instructed to fixate on the fixation target. The computer system superimposes the orientation grid onto the one or more images to obtain one or more output images. The one or more output images are displayed to at least one of a surgeon and the patient. The surgeon and/or patient may review the output images and communicate the location of floaters using coordinates of the orientation grid.

### TECHNICAL FIELD

The present disclosure relates generally to methods for treating vitreous floaters.

### BACKGROUND

Light received by the human eye, passes through the transparent cornea covering the iris and pupil of the eye. The light is transmitted through the pupil and is focused by a crystalline lens positioned behind the pupil in a structure called the capsular bag. The light is focused by the cornea and the lens onto the retina, which includes rods and cones capable of generating nerve impulses in response to the light. The space between the lens and the retina is occupied by a clear gel known as the vitreous.

Through various causes, floaters may be present in the vitreous. A floater is typically formed of a clump of cells or other tissue and is more opaque than the surrounding vitreous. Floaters cast shadows onto the retina that cause visual disturbance for a patient, which can be quite severe in some patients.

It would be an advancement in the art to facilitate the diagnosis and treatment of floaters.

### BRIEF SUMMARY

The present disclosure relates generally to a system for diagnosing and treating vitreous floaters.

Particular embodiments disclosed herein provide a method and corresponding apparatus, the method including projecting an orientation grid into an eye of a patient. A computer system captures one or more images of a retina of an eye of the patient while projecting the orientation grid into the eye of the patient. The computer system superimposes the orientation grid onto the one or more images to obtain one or more output images. The one or more output images are displayed to at least one of a surgeon and the patient.

The following description and the related drawings set forth in detail certain illustrative features of one or more embodiments.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 is a schematic representation of an eye having a floater.

FIG. 2A is a schematic diagram of a scanning laser ophthalmoscope (SLO).

FIG. 2B is a schematic diagram of a system for imaging a patient's retina with a fixation target in accordance with certain embodiments.

FIG. 3 is a fixation target with a polar coordinate grid for facilitating the diagnosis of floaters in accordance with certain embodiments.

FIGS. 4A is an SLO image of the retina and the shadow of the floater on the retina with a fixation target superimposed on the image in accordance with certain embodiments.

FIGS. 4B is an SLO image with a polar coordinate grid superimposed thereon in accordance with certain embodiments.

FIG. 5 is process flow diagram of a method for diagnosing vitreous floaters in accordance with certain embodiments.

FIG. 6 illustrates an example computing device that implements, at least partly, one or more functionalities for diagnosing floaters in accordance with certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

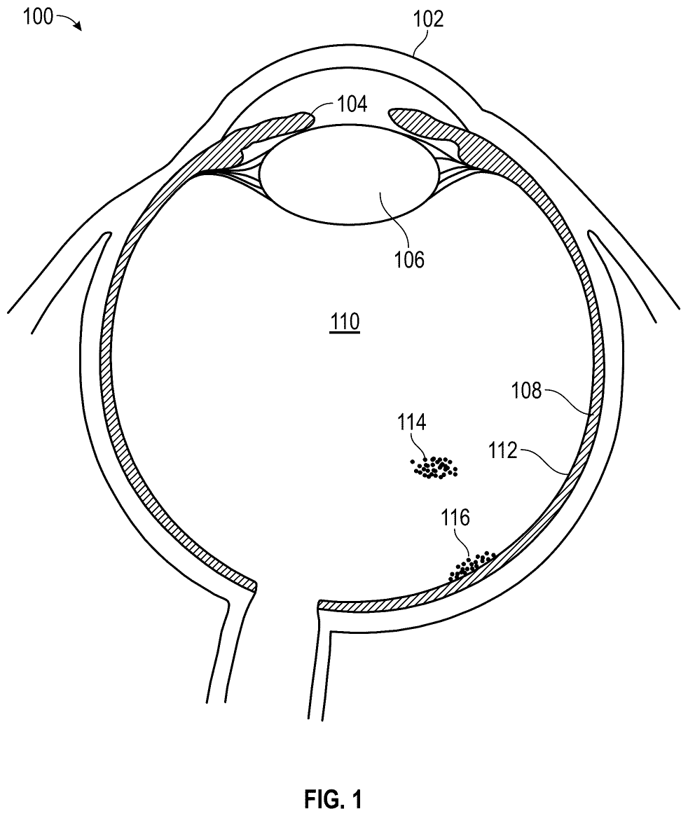

Referring to FIG. 1, a human eye 100 includes the cornea 102, which is a sphere-like transparent layer through which light enters the eye 100. The light then passes through the pupil 104 and lens 106 of the eye 100. The light is focused by the cornea 102 and lens 106 onto the retina 112 at the back of the eye 100. The remaining volume of the globe 108 of the eye 100 is occupied by a clear gel known as the vitreous 110.

The portion of the retina 112 across from the pupil 104 is known as the fovea and has the highest concentration of photoreceptor cells (rods and cones). The fovea occupies about 2 degrees of the field of view of the eye 100. The region around the fovea is known as the parafovea and the region around the parafovea is the perifovea. The size of the fovea, parafovea and perifovea are about 1.5 mm, 2.5 mm, and 5.5 mm respectively. For a normal eye, the visual angle is about equal to Arcsin (retinal distance measured in mm/17 mm). The angular sizes of the fovea, parafovea and perifovea are therefore 5.1°, 8.5° and 18.9° respectively.

Vitreous floaters 114 are clumps of cells, collagen fibers, or other deposits present in the vitreous 110. When present, a vitreous floater 114 will cast a shadow 116 onto the retina 112. The shadow 116 may occupy an angular extent of the field of vision of the eye 100. When sufficiently large, opaque, and/or numerous, floaters 114 can significantly reduce a patient's vision.

The floater is loosely embedded in the viscous vitreous. Upon usual visual activity, the eyeball rotates and the vitreous and the embedded floater and the shadow of the floater on the retina will move. This is the origin of the name of the floater. Sometimes floaters are called myodesopsias, i.e. flylike vision in Greek. The severity of vision degradation depends on many parameters such as the size of the floater 114, the optical density of the shadow 116, the distance of the shadow 116 from the fovea, the speed, and the direction of movement of the shadow 116. Typically, a shadow moving toward the fovea is more disturbing than a shadow moving away from the fovea. If the static position of the shadow 116 falls onto the fovea, the patient may experience irritation and loss of visual acuity. At the parafoveal and perifoveal locations, the moving shadows 116 do not influence the Snellen visual acuity but distract the visual attention of the patient. This attention-interrupting disturbance might be annoying or sometimes dangerous, such as when driving.

Due to the mechanical inertia of the gel-like vitreous 110, the movement of the shadow 116 is delayed with respect to the rotation of the eye 100. Upon fixation of the eye 100 on a stationary fixation target, the movement of the floater 114 stops after a few second and the shadow 116 will not move. Experiments conducted by the inventor have found that most floaters 114 have a permanent “home position.” Depending on the visual task, the shadow 116 of each floater 114 moves in the vicinity of this home position. The home position of a floater 114 and its corresponding shadow 116 typically remains unchanged for several months.

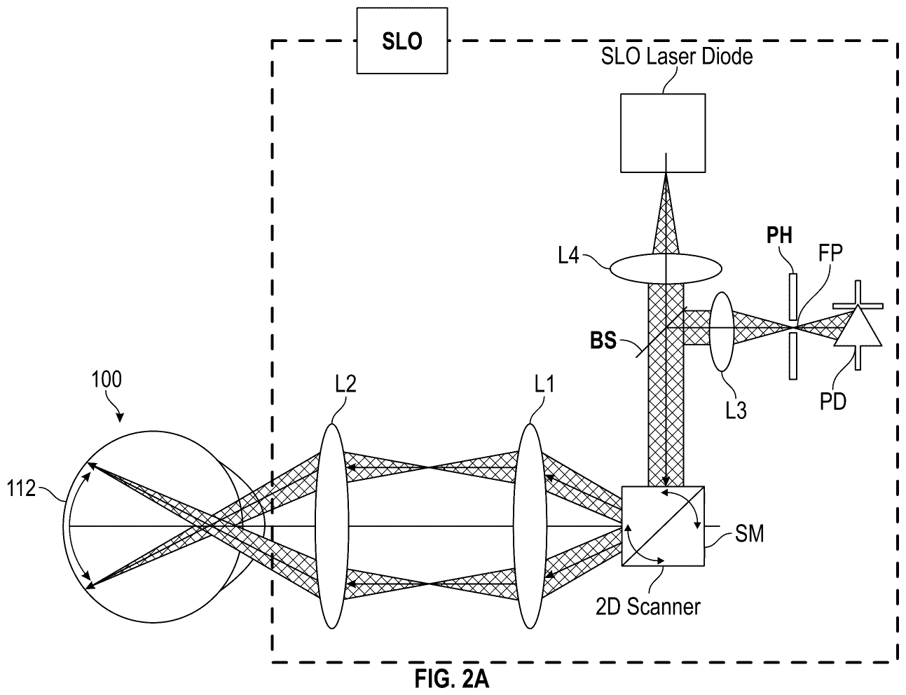

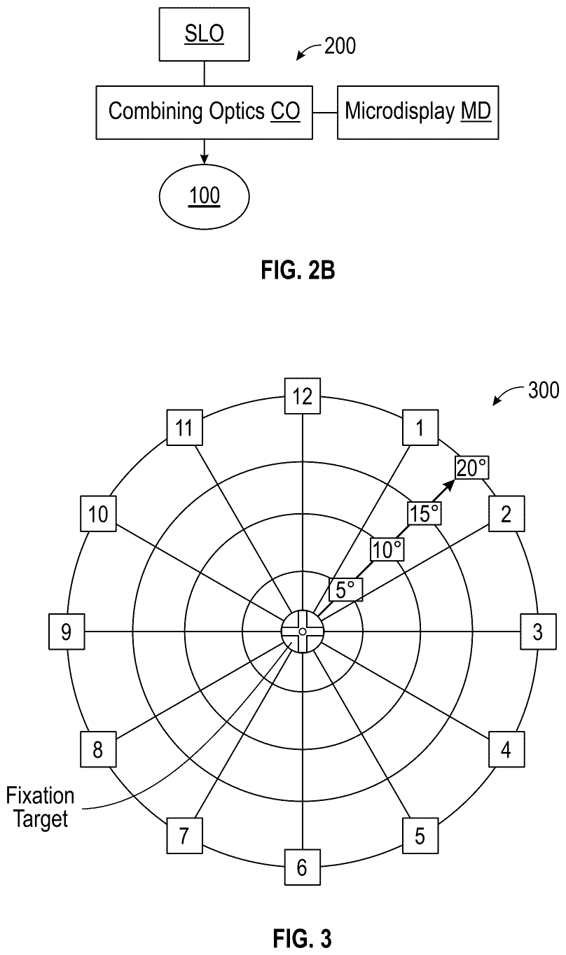

FIG. 2A illustrates an example SLO that may be used to implement the methods described herein. Although the system and methods are described herein as using an SLO, other imaging modalities may be used, such as an optical coherence tomography (OCT) device or fundus camera. The SLO includes a laser diode LD, which may be embodied as an infrared laser diode suitable for use in an SLO as known in the art. The infrared light from the laser diode LD is not visible to the patient.

The beam from the laser diode LD is made substantially (e.g., within 1 degree of) parallel using lens L4. The beam from the laser diode LD is incident on a scanning mirror SM that rotates about at least two rotational directions. For example, the scanning mirror SM may rotate in rotational directions RX and RY, which may be defined as rotation about the X and Y axes, respectively. In some implementations, the scanning mirror SM is implemented by a first mirror rotating about rotational direction RX (“the RX mirror”) and a second mirror rotating about direction RY (“the RY mirror”). For example, the RX mirror may be implemented as a resonant scanner whereas the RY mirror is implemented as a relatively slower galvo mirror.

Light reflected from the scanning mirror SM is directed through one or more lenses L1, L2, which focus the light on a focal point of the SLO, i.c., the retina 112 of the eye 100. A portion of the light from the focal point of the SLO is reflected back from the retina 112, passes back through the lenses L2, L1, and is descanned by the scanning mirror SM onto a beam splitter BS. The beam splitter BS directs at least a portion of the descanned light onto a photodiode PD. As is apparent from FIG. 2A, light emitted from the laser diode LD is incident on the beam splitter BS and a portion passes therethrough to reach the vitreous 110.

In some implementations, to reduce the detection of light reflected from the cornea, lens, or other structures located somewhere other than at the focal point of the laser diode LD on the retina, a lens L3 is positioned between the beam splitter BS and the photodiode PD. A pinhole PH is positioned in the focal point of the lens L3 between the lens L3 and the photodiode PD. In this way L3 and the pinhole PH forms a confocal filter suppressing any light which is not originated from the focal point of the laser diode LD on the surface of the retina 112. To have efficient depth selection, the diameter of the pinhole PH should be about (e.g., within 10 percent of) the diffraction limited spot diameter of the lens L3. The lens L3 may be implemented as a single lens or as a compound lens system.

The SLO may be coupled to a computer system, such as computer system having some or all of the attributes of the computing system 1000 described below. The computer system may receive the output of the photodiode PD continuously or at a sampling rate. The computer system may combine the output of the photodiode PD with the angular orientation of the mirrors from the encoder of RX and RY mirrors. Combining these data, the computer system can continuously create an en face 2D image of light reflected from the retina 112.

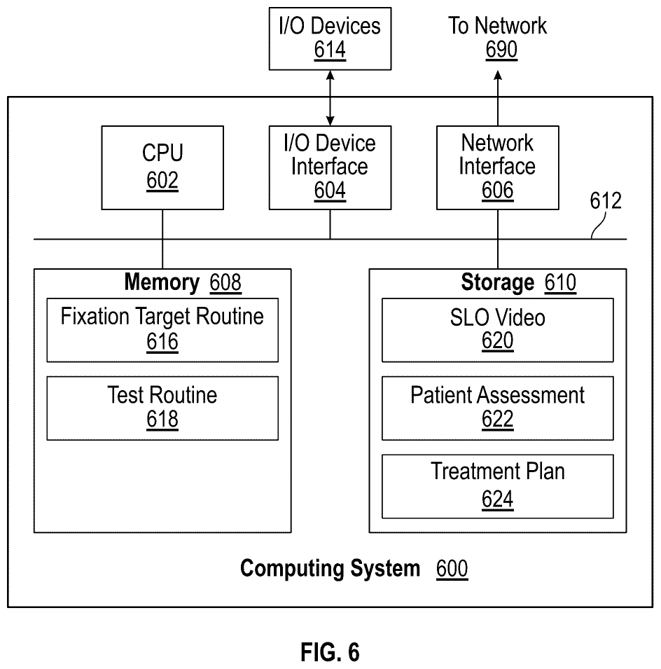

Referring to FIG. 2B, a system 200 may include an SLO combined with a microdisplay MD. The microdisplay MD projects a fixation target as described already above into the eye 100 of the patient and onto the retina 112. The microdisplay MD may be coupled to a computing system, such as a computing system 600 (see FIG. 6), which controls the image displayed on the microdisplay MD while capturing video from the SLO. Light from the SLO and the microdisplay MD may be combined by combining optics CO, such as one or more lenses, beam splitters, or other optical elements and directed into the eye 100 of the patient and focused on the retina 112.

Referring to FIG. 3, to assist a patient in identifying the angular position of a floater 114, a polar coordinate grid 300 may be displayed with the fixation target and projected onto the patient's retina 112 while capturing SLO video. The polar coordinate grid 300 may be annotated with the azimuthal angle either in degrees (e.g., a labeled radial line at 0, 30, 60, 90, 120, 150, 180, 210, 240, 270, 300, and 330 degrees) or in clock hour units (e.g., radial lines labeled 1 to 12 in FIG. 8). The radius in the polar coordinate grid 300 may be represented as concentric circles labeled with an angular offset from the visual axis of the eye 100 (e.g., 5°, 10°, 15° in FIG. 8). The geometrical size of the polar coordinate grid 300 image on microdisplay MD and any optics between the microdisplay MD and the patient's eye should be designed so that the angular offset labeling corresponds to the real visual angle of the patient. A Cartesian grid may also be used, such as a grid with horizontal lines labeled with numbers and vertical lines labeled with letters such that coordinates may be expressed as a letter and a number: A1, B5, etc. In either case, the grid is labeled with coordinates for two dimensions. Likewise, in either case, the center of the grid may include a fixation target.

The brightness of the fixation target, polar coordinate grid, or the background thereof should be bright enough to result in at least 1000 troland retinal illuminance, however values between 1000 and 5000 may also be used. At 1000 troland, the contrast sensitivity of the eye reaches the maximal sensitivity and therefore the floater shadow 116 is well visible for the patient and any vision disturbance is well noticeable. For a pupil having 1 mm² area, 1 troland is the retinal illuminance when a patient observes a surface having 1 cd/m². The typical luminance of a computer screen can be as high as 300 cd/m², giving 300 cd/m²*3.14 mm²=942 troland when looking onto a bright micro display with a pupil having 2 mm diameter. The pupil size typically exceeds 2 mm and therefore the 1000 troland retinal luminance is achievable with microdisplays.

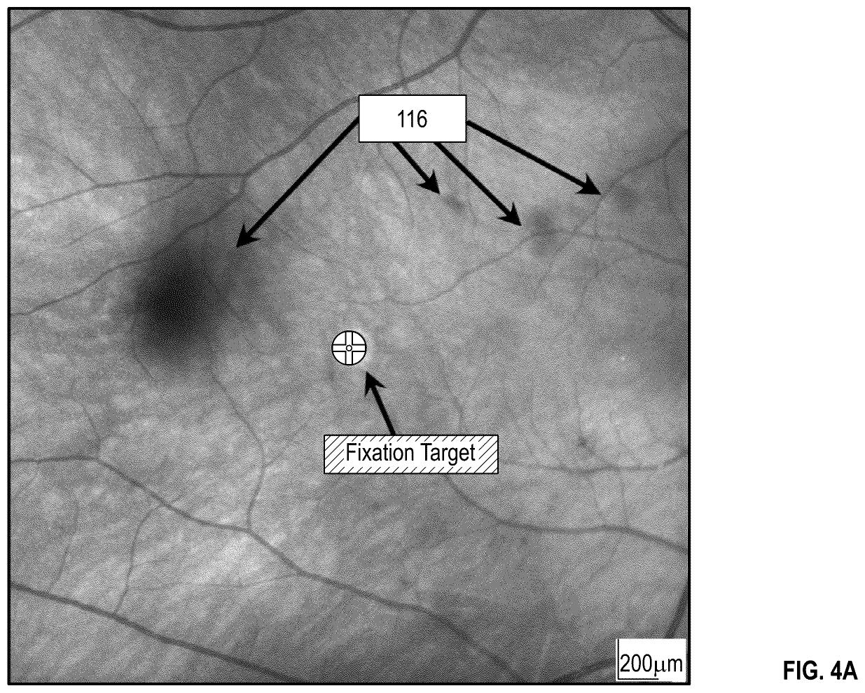

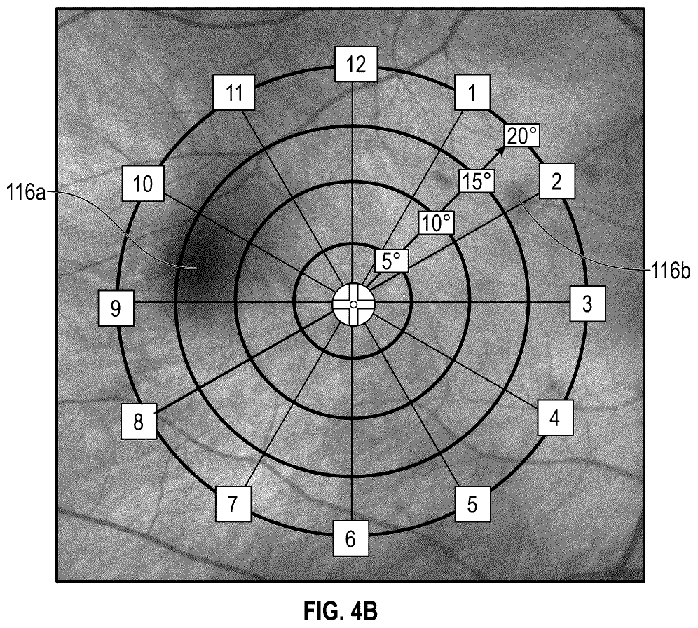

In the SLO, a fixation target is displayed to the patient and the patient is instructed to fix the gaze on the fixation target. FIG. 4A shows an SLO image of the retina with four shadows of four floaters as it appears on the display screen of the SLO. The fixation target is displayed for the patient in the visible spectral range. The SLO beam scans the retina with an infrared beam and the SLO is designed to not significantly detect the visible spectrum, therefore the image of the fixation target on the retina is not visible for the SLO. Software of the SLO may be configured to artificially superimpose the fixation target on the SLO image. In this way the surgeon is informed about the location of the fixation point of the patient during the measurement. FIG. 4A shows not only the retinal image with the four floater shadows but also the added image of the fixation target as displayed to the surgeon on the display of the SLO.

FIG. 4B illustrates an SLO image of FIG. 4A of a patient's retina having the polar coordinate grid 300 and fixation target of FIG. 3 superimposed thereon, i.c., the same image that was projected onto the patient's retina 112 when the SLO image of the patient's retina 112 was captured. During capture of the SLO image, retinal eye tracking may be performed to maintain the center of the SLO image at the center of the fovea of the patient. Retinal eye tracking may be performed using any approach known in the art of OCTs or SLOs. Prior to and/or during capture of the SLO image, the patient is instructed to center the patient's gaze on the center (e.g., center reticle) of the polar coordinate grid. The polar coordinate grid of FIG. 4B may be substantially (c.g., within 0.01 degrees) centered on the SLO image or otherwise centered on the center of the representation of the fovea in the SLO image. Accordingly, other than the illustrated retinal vasculature and other retinal features, the SLO image and superimposed polar coordinate grid approximate the visual experience of the patient when the SLO image was captured, assuming that the patient approximately centered the patient's gaze on the center of the polar coordinate grid.

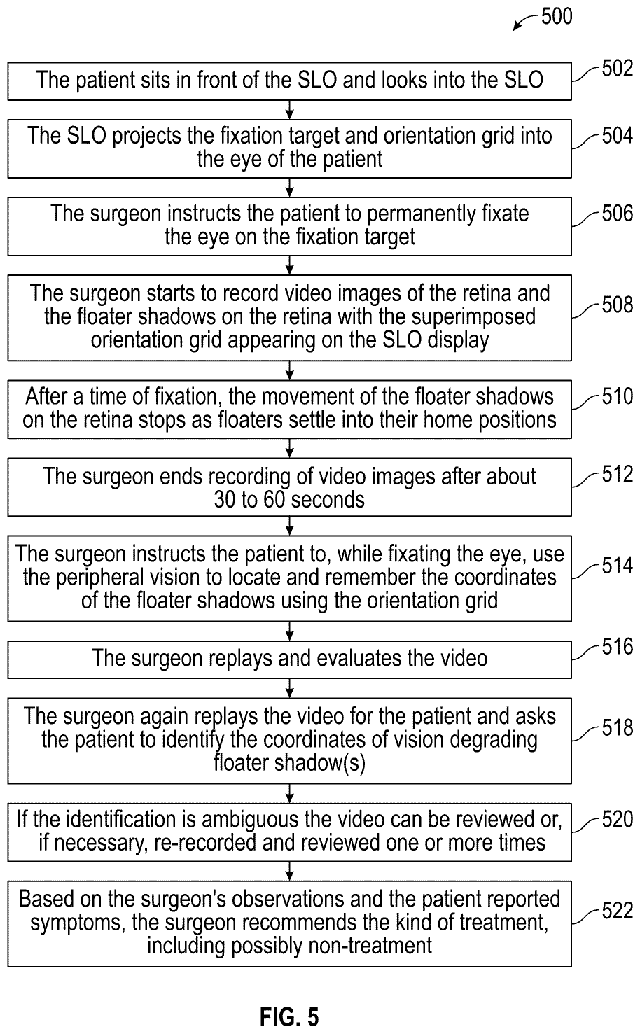

FIG. 5 illustrates a process flow diagram of a method 500 for diagnosing vitreous floaters. The method 500 may be performed by a computing system 600 with cooperation of a patient and an operator, such as a surgeon or other health professional.

At step 502, the patient sits in front of the SLO and looks into the SLO. At step 504, the SLO projects the fixation target and an orientation grid (c.g., polar coordinate grid 300) into the eye of the patient. At step 506, the surgeon, or instructions displayed on the microdisplay MD or audibly played back by the SLO itself, instructs the patient to permanently fixate the eye on the fixation target. At step 508, the surgeon starts to record video images of the retina, any floater shadows on the retina while the fixation target and orientation grid are displayed to the patient and the patient continues to fixate on the fixation target. Eye tracking may be performed during recording or may be omitted in view of the patient fixation on the fixation target. The video images may be displayed on a display device having the orientation grid and fixation target superimposed thercon. At step 510, after a few seconds of fixation, the movement of the floater shadows on the retina stops as the floaters settle into their home positions. At step 512 the surgeon ends recording, such as after about 30 to 60 seconds or some other duration.

At step 514, the surgeon instructs the patient to, while fixating the eye, use the peripheral vision to locate and remember the coordinates of the floater shadows using the orientation grid. For example, 9:30 and about 15° and 2:00 and about 15° for the shadows of FIG. 4B. At step 516, the surgeon replays and medically evaluates the video, which may include the surgeon identifying the coordinates and possibly size of floater shadows apparent in the video. The video may be displayed on a separate display device, such as a computer screen or television.

At step 518, the surgeon again replays the video for the patient and asks the patient to identify the coordinates of vision degrading floater shadow or shadows. Step 518 is preferably performed immediately after performing step 506, e.g., within 2 minutes, such that the patient remembers the coordinates of any shadows 116 perceived. The patient may identify the coordinates by verbally speaking or typing in coordinates of the orientation grid. The coordinates may also be provided by receiving a user input to a touch screen. For example, the SLO video or still images with the superimposed fixation target may be displayed on a touch screen and the patient may tap points on the screen that the patient believes correspond to floaters. The user may provide additional inputs for each floater shadow 116a, 116b identified, such as a rating of severity (e.g., 1 indicating barely noticeable and 10 indicating substantial interference with vision) or an estimate of size. This input may be provided verbally, typed in, input through an interface provided on the touch screen.

At step 520, if the identification by the patient is ambiguous, the video can again be reviewed or if necessary can be re-recorded and reviewed one or more times so long as the identification is unambiguous. At step 522, based on the surgeon observations and the patient reported symptoms the surgeon recommends the kind of the treatment, which may include a recommendation of non-treatment. Step 520 may include entering a proposed course of treatment to the computing system 600. The proposed course of treatment may be stored along with some or all of the SLO video or still images recorded at step 508, the SLO video or still images with the fixation target superimposed thereon, and the patient identification received at step 518.

FIG. 6 illustrates an example computing system 600 that implements, at least partly, one or more functionalities described herein with respect to FIGS. 3 to 5. The computing system 600 may be integrated with an imaging device, such as the SLO, or be a separate computing device receiving images of a patient's eye from the SLO and controlling fixation targets displayed on the microdisplay MD.

As shown, computing system 600 includes a central processing unit (CPU) 602, one or more I/O device interfaces 604, which may allow for the connection of various I/O devices 614 (e.g., keyboards, displays, mouse devices, pen input, etc.) to computing system 600, network interface 606 through which computing system 600 is connected to network 690, a memory 608, storage 610, and an interconnect 612.

In cases where computing system 600 is an imaging system, such the SLO, an OCT, or fundus camera, the computing system 600 may further include one or more optical components for obtaining ophthalmic imaging of a patient's eye as well as any other components known to one of ordinary skill in the art.

CPU 602 may retrieve and execute programming instructions stored in the memory 608. Similarly, CPU 602 may retrieve and store application data residing in the memory 608. The interconnect 612 transmits programming instructions and application data, among CPU 602, I/O device interface 604, network interface 606, memory 608, and storage 610. CPU 602 is included to be representative of a single CPU, multiple CPUs, a single CPU having multiple processing cores, and the like.

Memory 608 is representative of a volatile memory, such as a random access memory, and/or a nonvolatile memory, such as nonvolatile random access memory, phase change random access memory, or the like. As shown, memory 608 may store fixation target routine 616 configured to control display of the fixation target and polar coordinate grid 300 on the microdisplay MD. The memory may further store a test routine 618 including executable code for performing the computerized steps of the method 500 as described above.

Storage 610 may be non-volatile memory, such as a disk drive, solid state drive, or a collection of storage devices distributed across multiple storage systems. Storage 610 may optionally store SLO video 620 captured at step 506 of the method 500, a patient identification 622 received at step 518, and/or a treatment plan 624 received at step 522.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112 (f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method comprising:
projecting an orientation grid into an eye of a patient;
capturing, by a computer system, one or more images of a retina of an eye of the patient while projecting the orientation grid into the eye of the patient;
superimposing, by the computer system, the orientation grid onto the one or more images to obtain one or more output images; and
displaying by the computer system, the one or more output images to at least one of a surgeon and the patient.

2. The method of claim 1, wherein the orientation grid is a polar coordinate grid.

3. The method of claim 2, wherein azimuthal angle lines of the polar coordinate grid are annotated with hour labels 1 to 12.

4. The method of claim 1, further comprising displaying a fixation target along with the orientation grid.

5. The method of claim 4, further comprising instructing the patient to fixate on the fixation target.

6. The method of claim 4, wherein capturing the one or more images of the retina comprises detecting infrared light reflected from the retina while the patient fixates on the fixation target.

7. The method of claim 6, wherein capturing the one or more images comprises capturing one or more images of the retina using a scanning laser ophthalmoscope (SLO).

8. The method of claim 1, wherein capturing the one or more images comprises capturing video of the retina.

9. The method of claim 1, further comprising, receiving, by the computer system, patient selection of coordinates in the orientation grid corresponding to one or more angular locations of one or more shadows on the retina.

10. The method of claim 9, further comprising, receiving, by the computer system, a treatment plan for one or more vitreous floaters in the eye.

11. A system comprising:
an imaging device configured to both (a) project an orientation grid into the eye of a patient, the orientation grid having coordinate labels in two angular dimensions (b) capture one or more images of a retina of the eye of the patient during (a); and
a computer system coupled to the imaging device and configured to:
superimpose the fixation target and the orientation grid onto the one or more images to obtain one or more output images; and
display the one or more output images to at least one of a surgeon and the patient.

12. The system of claim 11, wherein the orientation grid is a polar coordinate grid.

13. The system of claim 12, wherein azimuthal angle coordinates of the polar coordinate grid are annotated with hour labels 1 to 12.

14. The system of claim 11, wherein the imaging device is configured to display a fixation target along with the orientation grid at (a).

15. The system of claim 14, wherein the computer system is further configured to audibly instruct the patient to fixate on the fixation target.

16. The system of claim 14, wherein the imaging device is an SLO configured to capture the one or more images of the retina by detecting infrared light reflected from the retina while the patient fixates on the fixation target.

17. The system of claim 16, wherein the imaging device is configured to perform (a) by projecting the orientation grid as visible light.

18. The system of claim 11, wherein the imaging device is configured to capture the one or more images by capturing video of the retina.

19. The system of claim 11, wherein the computer system is further configured to receive patient selection of coordinates in the orientation grid corresponding to one or more angular locations of one or more shadows on the retina.

20. The system of claim 19. wherein the computer system is further configured to receive a treatment plan for one or more vitreous floaters in the eye of the patient.
